(19) United States
(12) Patent Application Publication
(10) Pub. No.: US 2023/0200876 A1
(43) Pub. Date: Jun. 29, 2023

(54) WRIST FUSION SYSTEM AND DELIVERY TOOL

(71) Applicant: ExsoMed Corporation, Aliso Viejo, CA (US)

(72) Inventors: Martin Boyer, St. Louis, MO (US); Kathleen McKeon, Birmingham, AL (US); Kavi Sachar, Englewood, CO (US); Andrew J. Leither, Akron, OH (US); Michael Zwolinski, Willoughby, OH (US)

(21) Appl. No.: 17/923,449

(22) PCT Filed: Apr. 30, 2021
(86) PCT No.: PCT/US2021/030162

§ 371 Date: Nov. 4, 2022

Related U.S. Application Data
(60) Provisional application No. 63/021,386, filed on May 7, 2020.

(51) Int. Cl. A61B 17/88; A61B 17/80; A61B 17/86

(52) U.S. Cl. CPC A61B 17/8866; A61B 17/8061; A61B 17/8057; A61B 17/86; A61B 2017/564

(57) ABSTRACT

A four corner fusion system can include a curved plate that is more aligned with the natural anatomy of the wrist joint, and/or fewer number of screws (for example, about three or four screws). The system can have a lower profile, resulting in less soft tissue impingement. A surgical tool with a pair of offsetting clamping arms can be used to aid in bone reduction during a wrist fusion procedure.

### INCORPORATION BY REFERENCE TO ANY PRIORITY APPLICATIONS

This application claims the priority benefit of U.S. Provisional Application Nos. 63/021,386, filed May 7, 2020, the entirety of which is hereby incorporated by reference herein and should be considered part of this specification. Any and all applications for which a foreign or domestic priority claim is identified in the Application Data Sheet as filed with the present application are hereby incorporated by reference under 37 CFR 1.57.

### OTHER RELATED APPLICATIONS

U.S. Pat. App. Pub. No. 2019/0307570 (published on Oct. 10, 2019) describes various example embodiments and features related to apparatuses, systems, surgical tools, and methods of performing partial wrist fusion. U.S. Pat. App. Pub. No. 2019/0307570 is incorporated in its entirety by reference and is part of this disclosure. The embodiments described below are compatible with and can be part of the embodiments described in U.S. Pat. App. Pub. No. 2019/0307570, and some or all of the features described below can be used or otherwise combined together or with any of the features described in U.S. Pat. App. Pub. No. 2019/0307570.

### BACKGROUND

The present disclosure relates to partial wrist fusion, for example, by using the four corner fusion technique.

Patients with wrist pain and/or arthritis may need surgical intervention. One example surgical procedure for such patients is the four corner fusion technique, which involves excision or removal of the scaphoid bone and fixation of the remaining wrist bones with a plate or wires.

Existing four corner fusion systems can include a fixation plate and a plurality of screws (for example, about 6 to 8 screws) to achieve four corner fusions.

### SUMMARY

The present disclosure provides an example surgical tool configured to be used in wrist fusion, or intercarpal fusion, or four corner fusion. The tool can comprise first and second clamping arms that are each offset relative to a transverse plane defined by a main body of the tool; and a locking mechanism for locking relative positions of the first and second clamping arms once a desired alignment of bones at a wrist joint is achieved.

In a configuration, the first and second clamping arms can be offset relative to the transverse plane in opposite directions.

In a configuration, the first and second clamping arms can be offset relative to the transverse plane such that the tool is configured to be for use by a left-handed user.

In a configuration, the first and second clamping arms can be offset relative to the transverse plane such that the tool is configured to be for use by a right-handed user.

In a configuration, the locking mechanism can comprise a screw.

The present disclosure provides an example surgical method for performing wrist fusion on a patient using a plate having a curvature configured to substantially follow the natural anatomy of a wrist joint in combination with a reduction tool with offset first and second clamping arms. The method can comprise attaching the plate to bone(s) at the wrist joint of the patient using at least one screw; using the reduction tool to reduce the bones at the wrist joint by rotating the offset clamping arms to push a capitate at the wrist joint toward a palm of the patient's hand connected to the wrist joint; and inserting a remainder of a plurality of screws into the bones at the wrist joint, wherein, when fully implanted, the plurality of screws are coupled to the plate and inserted into the bones at the wrist joint in different directions.

In a configuration, using the reduction tool can be performed before attaching the plate to the bone(s) using at least one screws.

In a configuration, using the reduction tool can be performed after attaching the plate to the bone(s) using at least one screws.

In a configuration, the first and second clamping arms c offset relative to a transverse plane defined by a main body of the tool in opposite directions.

In a configuration, the first and second clamping arms of the reduction tool can be offset relative to the transverse plane such that the tool is configured to be for use by a left-handed user.

In a configuration, the first and second clamping arms of the reduction tool can be offset relative to the transverse plane such that the tool is configured to be for use by a right-handed user.

In a configuration, the method can further comprise locking relative positions of the first and second clamping arms once a desired alignment of bones at the wrist joint is achieved using a locking mechanism.

In a configuration, the locking mechanism can comprise a screw.

The present disclosure provides an example system for fusing at least two bones in a wrist, or intercarpal fusion, or four corner fusion. The system can comprise a plate having a curvature configured to substantially follow the natural anatomy of the wrist; and a plurality of screws configured to point in different directions when coupled to the plate.

In a configuration, the plurality of screws can comprise locking screws.

In a configuration, the plurality of screws can comprise non-locking screws.

### BRIEF DESCRIPTION OF THE DRAWINGS

These and other features, aspects, and advantages of the present disclosure are described with reference to the drawings of certain embodiments, which are intended to schematically illustrate certain embodiments and not to limit the disclosure.

FIGS. 1A-1C illustrate various views of an example four corner fusion system implanted in a wrist joint.

FIGS. 2A-2D illustrate various views of a fixation plate of the four corner fusion system of FIG. 1A.

FIGS. 3A-3C illustrate various views of the assembled four corner fusion system of FIG. 1A.

FIGS. 4A-4B illustrate various views of a locking screw of the four corner fusion system of FIG. 3A.

FIGS. 5A-5B illustrate various views of a non-locking screw of the four corner fusion system of FIG. 3A.

FIGS. 6A-6C illustrate various views of an example surgical tool for use in a partial wrist fusion procedure.

FIGS. 7A-7B illustrate various views of an example surgical tool applied to a wrist joint.

FIG. 8A illustrates an example X-ray image of a plurality of surgical tools for delivering a four corner fusion system to a wrist joint.

FIG. 8B illustrates a perspective view of a plurality of surgical tools for delivering a four corner fusion system to a wrist joint.

FIG. 9 illustrates another example four corner fusion system implanted in a wrist joint.

### DETAILED DESCRIPTION

Although certain embodiments and examples are described below, those of skill in the art will appreciate that this disclosure extends beyond the specifically disclosed embodiments and/or uses and obvious modifications and equivalents thereof. For example, the example wrist fusion system may also be implanted in other parts of the human body, such as the foot. Thus, it is intended that the scope of the disclosure herein disclosed should not be limited by any particular embodiments described below.

Existing four corner fusion systems can have a relatively large profile, for example, due to the shape of the fixation plate, the number of screws used, and/or the orientation of those screws, resulting in significant soft tissue impingement.

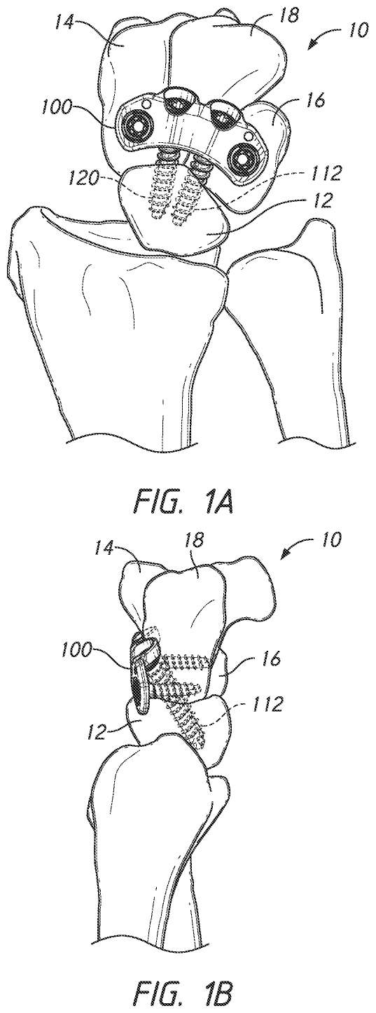

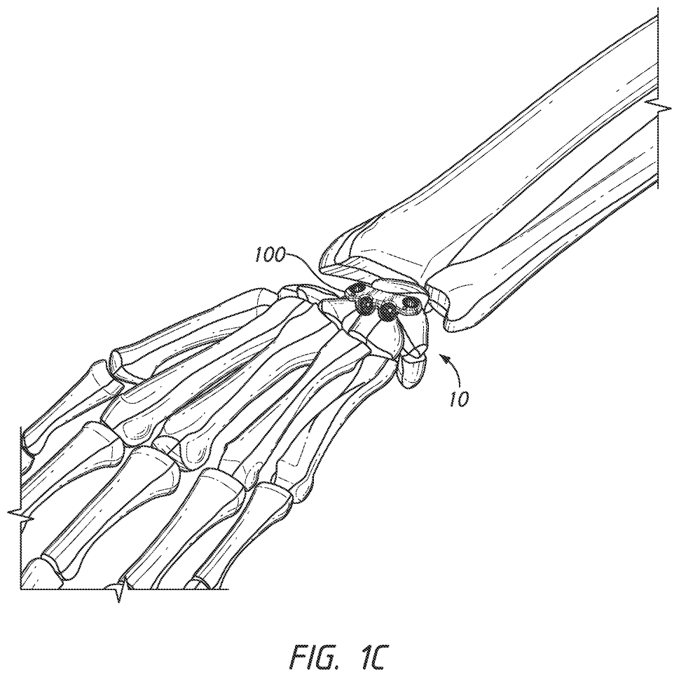

The four corner fusion system of the present disclosure can have a lower profile, resulting in less soft tissue impingement. An example fusion system 100 that is implanted in a wrist joint 10 is shown in FIGS. 1A-1C. In FIGS. 1A and 1B, the wrist joint 10 is illustrated with the scaphoid bone removed. The fusion system 100 can include a curved plate that is more aligned with the natural anatomy of the wrist joint 10, and/or fewer number of screws (for example, about three or four screws). When the screws are tightened, the plate aligns better with the natural anatomy of the wrist joint 10. The screws can include non-locking screws, fixed-angle locking screws, and/or variable-angle locking screws.

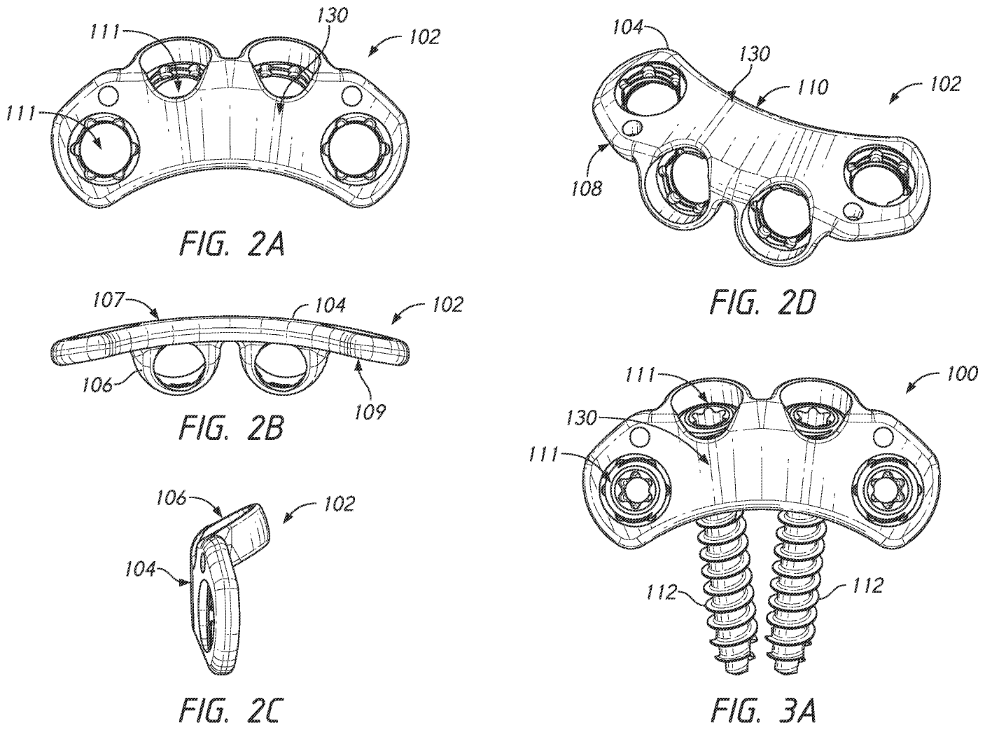

FIGS. 2A-2D illustrates a plate 102 of the four corner fusion system 100 of FIGS. 1A-1C. When implanted, the shape and/or dimension of the plate 102 can better conform to the natural anatomy of the wrist joint. The plate 102 can have a length sufficient to span across the remaining wrist bones after removal of the scaphoid bone. For example, the length can be between about 10 cm to about 35 cm, or between about 15 cm to about 30 cm, or between about 22 cm to about 26 cm.

As shown in FIGS. 2B and 2C, the plate 102 can include a first portion 104 and a second portion 106. As shown in FIG. 2D, the first portion 104 can have a first side 108 that curves outwardly relative to a body of the first portion 104. The first portion 104 can have a second side 110 opposite the first side 108. The second side 110 can curve inwardly relative to the body of the first portion 104. As shown in FIGS. 1A-1C, when implanted, the first side 108 is the distal side (closer to the hand) and the second side 110 is the proximal side (closer to the arm). The curvature of the first and second sides 108, 110 can reduce the likelihood of the implanted plate 102 being in the way of the bones of the hand, wrist, and/or arm during movement of the wrist joint.

As shown in FIG. 2B, the body of the first portion 104 can be curved in a direction generally transverse to the curvatures of the first and second sides 108, 110. When implanted, a concave side 109 of the body of the first portion 104 can be facing the wrist joint and the convex side 107 of the body of the first portion 104 can be facing away from the wrist joint. The curvature in the body of the first portion 104 can better align with the anatomical curvature of the wrist joint than a fusion plate that has a reversed curvature when implanted, that is, with the convex side facing the wrist joint.

The second portion 106 can be coupled to the first portion 104 on a first side 108 of the first portion 104. As shown in FIG. 2C, the first and second portions 104, 106 can be at an angle with each other. As shown in FIGS. 1B and 1C, the angle can allow the first and second portions 104, 106 to better align with the surface of the fused wrist bones. In some embodiments, the angle can be between about 15° to about 45°, or between about 25° to about 35°, or between about 28° to about 32°.

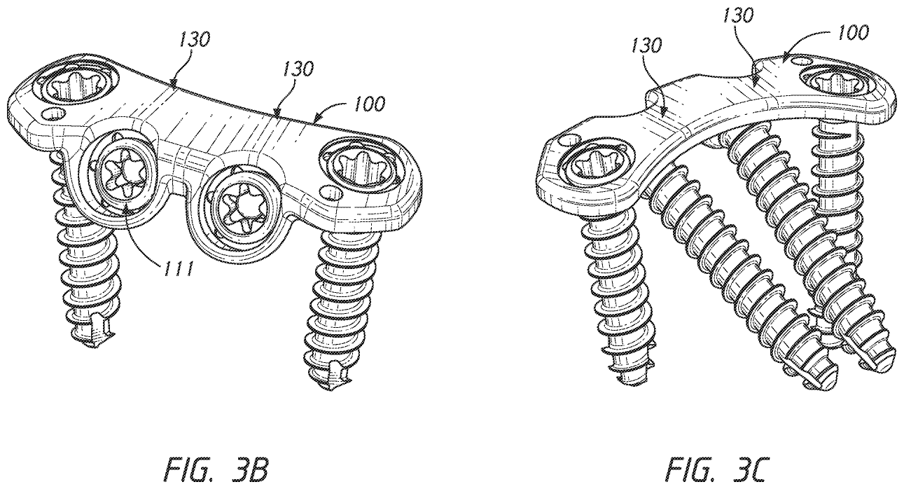

As shown in FIGS. 2A-3C, the plate 102 of the fusion system 100 of FIGS. 1A-1C can include a plurality of openings 111 configured to accommodate a plurality of screws 112. The openings 111 can have varying orientations such that the screws 112 can point in different directions when assembled with the plate 102. For example, the screws 112 coupled to the openings 111 of the first portion 104 can be directed to a first direction that is generally at an angle to a second direction of the screws 112 coupled to the openings 111 of the second portion 106. The size, number, and/or location of the openings 111 can vary. Having the screws 112 pointing in different directions can allow the screws 112 to be directed to the different bones at the wrist joint. In the illustrated embodiment such as shown in FIGS. 1A and 1B, the screws 112 coupled to the openings of the second portion can be directed to and be inserted into the lunate 12 after the screws 112 have penetrates the other bones at the wrist joint which are located more distally than the lunate 12, such as the capitate 14, the hamate 18, and/or the triquetrum 16.

With continued reference to FIGS. 2A, 2D, and 3A-3C, the convex side 107 of the body of the first portion 104 can include a plurality of markings 130 extending for at least partially a width of the first portion 104, for example, extending substantially from the first end 108 to the second end 110. The markings can extend to or close to the openings 111 of the second portion 106 and can indicate the direction of the screws 112 (and/or aid in visualization of the screws 112) coupled to the openings 111 of the second portion. The markings can be etched by laser or made by other suitable methods.

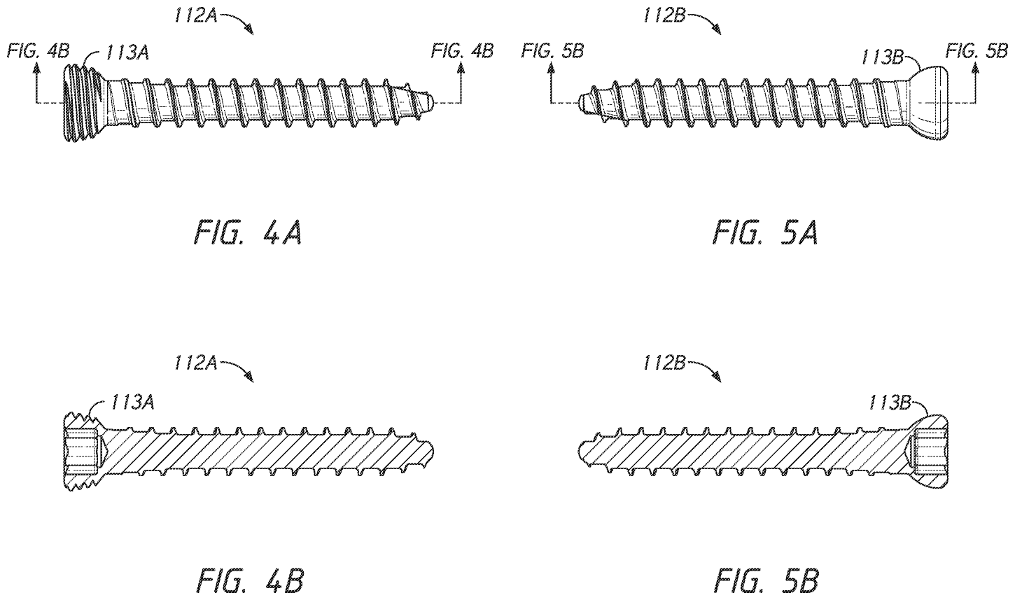

The screws can be locking 112A (FIGS. 4A-4B) or non-locking 112B (FIGS. 5A-5B). The locking screw 112A can include a threaded screw head 113A. The non-locking screw 112B can include a non-threaded screw head 113B. The terms “locking” and “non-locking” have their plain meanings as understood by a person of ordinary skill in the art. The pitch, diameter and/or length of the screws can vary. In some embodiments, the diameter of the locking and/or non-locking screws can be between about 1.5 mm to about 3.5 mm, or between about 2 mm to about 2.5 mm, or between about 3.0 mm to about 3.3 mm. In some embodiments, the length of the locking and/or non-locking screws can be between about 5 mm to about 40 mm, or between about 6 mm to about 36 mm, or between about 6 mm to about 12 mm, or between about 10 mm to about 18 mm, or between about 16 mm to about 24 mm, or between 20 mm to about 30 mm.

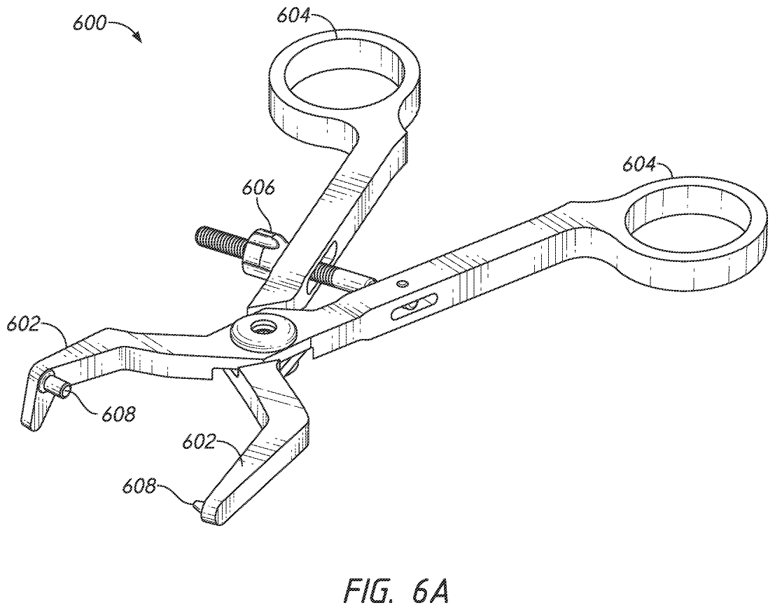

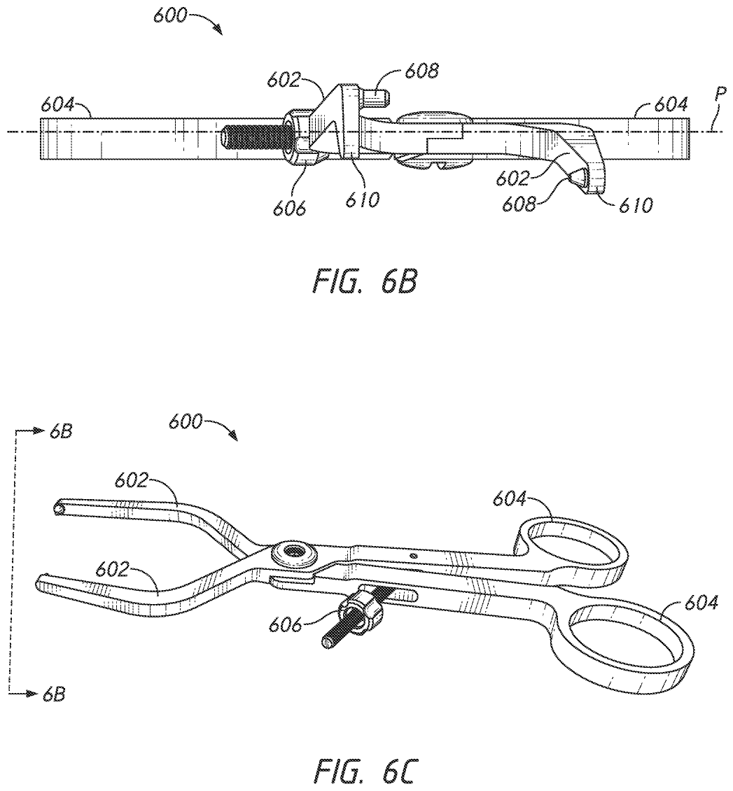

The present disclosure includes a surgical tool that can be used for a partial wrist fusion procedure. The tool can be used for delivering the four corner fusion system disclosed herein and/or any other partial wrist fusion systems. An example of the tool 600 is shown in FIGS. 6A-6C. The tool 600 can include two offset clamping arms 602. For example, as shown in FIG. 6B, free ends 610 of the arms 602 can be located on opposite sides of a plane P, which can be a central transverse plane across gripping portions 604 of the tool 600. The gripping portions 604 (for example, rings for gripping by one or more fingers or otherwise) can be configured to allow a user (for example, a surgeon, a nurse, or otherwise) to adjust a distance between the two offset clamping arms 602. Each arm 602 can include a protrusion 608 at or near its free end. The protrusions 608 on the arms 602 can generally face each other. The protrusions 608 can aid in gripping bones by the arms 602.

During a four corner fusion procedure, the bones associated with the wrist joint tend to shift (for example, shifting medially) after the scaphoid bone is removed. Currently, reduction of the bones or the natural alignment of the bones may need to be achieved directly by hand during implantation of a fusion device. The surgeon may need to use one hand to maintain the alignment of the bones and the other hand to implant the plate and screws. Alternatively, an assistant's hand(s) may be required to maintain the alignment of the bones as the surgeon secures a fusion device to the wrist joint. As described below, the tool 600 can be used to maintain alignment of the bones at the wrist joint without an external independent force. The tool 600 can thus free the surgeon's hand and/or remove the need for an assistant to maintain the alignment of the bones during the four corner fusion procedure.

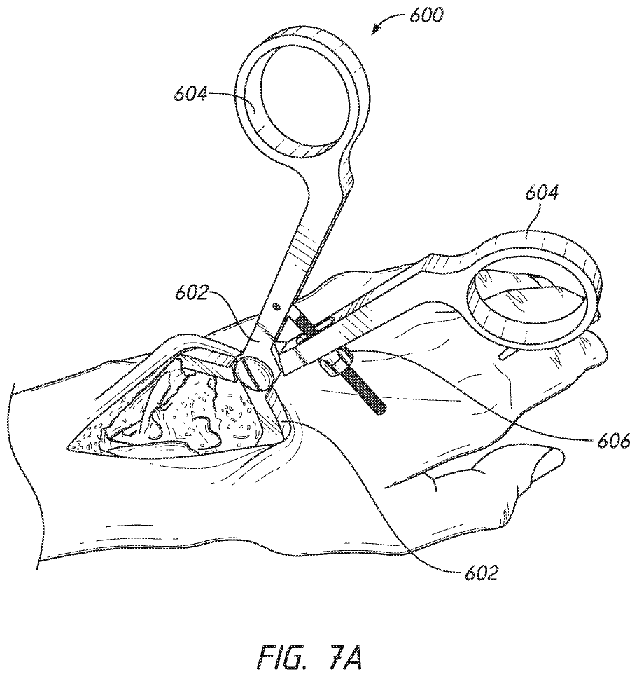

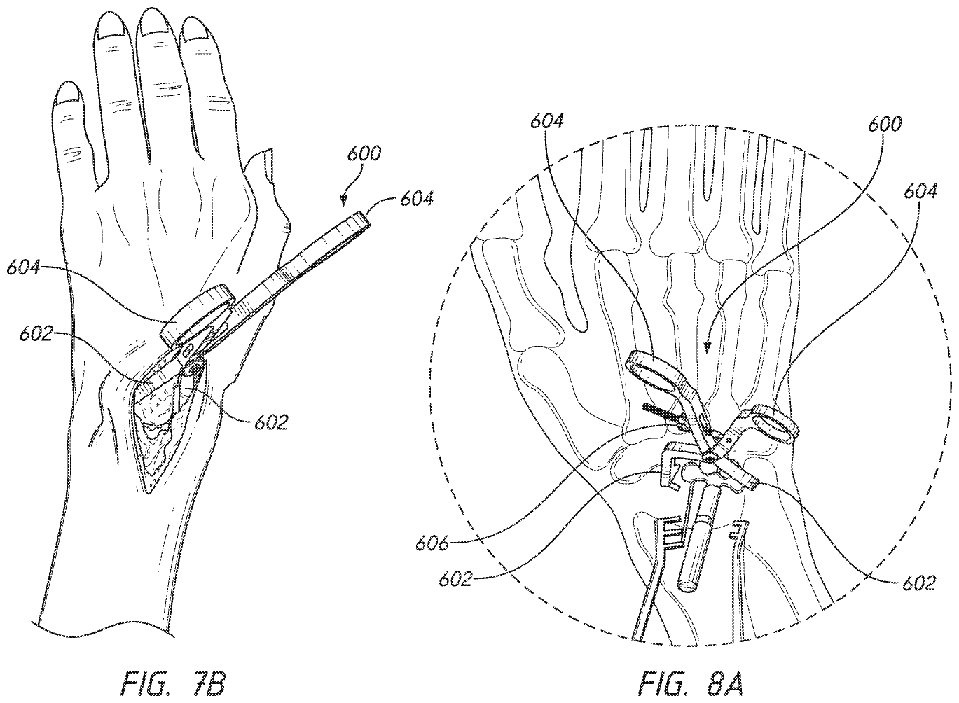

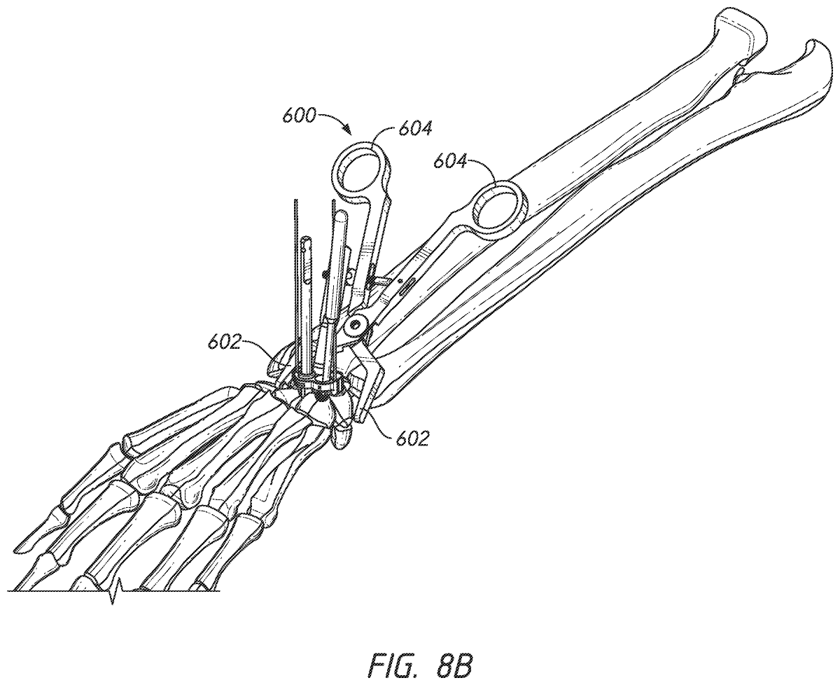

As shown in FIGS. 7A-8B, the clamping arms 602 can grab onto the bones at the wrist joint to reduce and/or prevent shifting of the wrist bones. Once the desired amount of reduction is achieved, the relative position of the two clamping arms 602 can be locked by a locking nut 606. The reduction maneuver using the clamp 600 includes rotating the clamp 600, therefore the clamping arms 602, to push the capitate toward the palm of the hand. The reduction maneuver can be done after a first screw insertion (for example, into the triquetrum or otherwise) or prior to any screw insertion. The gripping portions 604 of the tool 600 can be biased (for example, using a spring) to keeping the distance between the arms 602, for example, the distance between the free ends of the arms 602 at a predetermined value. The locking nut 606 can lock the relative positions of the arms 602, such as indirectly by locking the relative positions of the gripping portions 604, so that the locked distance between the free ends of the arms 602 is smaller than the predetermined value. As the distance between the free ends of the arms 602 is reduced, the bones that have shifted can be brought back closer to their naturally occurring positions before the scaphoid bone is removed. The offset in the arms 602 can make it easier for the user to rotate the gripped wrist bones, which can further facilitate reduction of the bones at the wrist joint.

The surgical tool 600 can be left-handed or right-handed. In some embodiments, the orientation of the free ends of the clamping arms 602 relative to the plane P can be reversed when comparing a left-handed tool 600 with a right-handed tool 600. In some embodiments, a user can use the same hand to deploy a left-handed tool 600 or a right-handed tool 600 to perform a four corner fusion procedure on a left hand or a right hand of a patient, respectively.

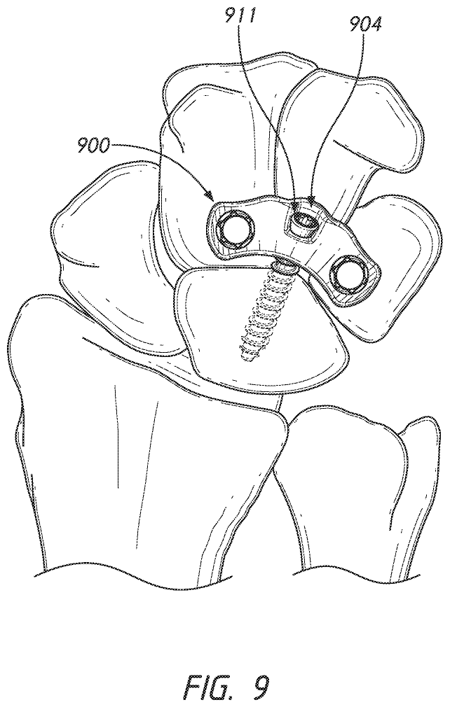

Another example of a four corner fusion system of the present disclosure is illustrated in FIG. 9. FIG. 9 illustrates a fusion system 900 with a plate that can have any of the features of the plate 102 disclosed herein, except that a second portion 904 of the plate has a different number of opening for receiving a screw than the plate 102. For example, the second portion 904 may have a single opening 911 as shown in FIG. 9.

Although this disclosure has been described in the context of certain embodiments and examples, it will be understood by those skilled in the art that the disclosure extends beyond the specifically disclosed embodiments to other alternative embodiments and/or uses and obvious modifications and equivalents thereof. In addition, while several variations of the embodiments of the disclosure have been shown and described in detail, other modifications, which are within the scope of this disclosure, will be readily apparent to those of skill in the art. It is also contemplated that various combinations or sub-combinations of the specific features and aspects of the embodiments may be made and still fall within the scope of the disclosure. For example, features described above in connection with one embodiment can be used with a different embodiment described herein and the combination still fall within the scope of the disclosure. It should be understood that various features and aspects of the disclosed embodiments can be combined with, or substituted for, one another in order to form varying modes of the embodiments of the disclosure. Thus, it is intended that the scope of the disclosure herein should not be limited by the particular embodiments described above. Accordingly, unless otherwise stated, or unless clearly incompatible, each embodiment of this invention may comprise, additional to its essential features described herein, one or more features as described herein from each other embodiment of the invention disclosed herein.

Features, materials, characteristics, or groups described in conjunction with a particular aspect, embodiment, or example are to be understood to be applicable to any other aspect, embodiment or example described in this section or elsewhere in this specification unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The protection is not restricted to the details of any foregoing embodiments. The protection extends to any novel one, or any novel combination, of the features disclosed in this specification (including any accompanying claims, abstract and drawings), or to any novel one, or any novel combination, of the steps of any method or process so disclosed.

Furthermore, certain features that are described in this disclosure in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations, one or more features from a claimed combination can, in some cases, be excised from the combination, and the combination may be claimed as a subcombination or variation of a subcombination.

Moreover, while operations may be depicted in the drawings or described in the specification in a particular order, such operations need not be performed in the particular order shown or in sequential order, or that all operations be performed, to achieve desirable results. Other operations that are not depicted or described can be incorporated in the example methods and processes. For example, one or more additional operations can be performed before, after, simultaneously, or between any of the described operations. Further, the operations may be rearranged or reordered in other implementations. Those skilled in the art will appreciate that in some embodiments, the actual steps taken in the processes illustrated and/or disclosed may differ from those shown in the figures. Depending on the embodiment, certain of the steps described above may be removed, others may be added. Furthermore, the features and attributes of the specific embodiments disclosed above may be combined in different ways to form additional embodiments, all of which fall within the scope of the present disclosure. Also, the separation of various system components in the implementations described above should not be understood as requiring such separation in all implementations, and it should be understood that the described components and systems can generally be integrated together in a single product or packaged into multiple products.

For purposes of this disclosure, certain aspects, advantages, and novel features are described herein. Not necessarily all such advantages may be achieved in accordance with any particular embodiment. Thus, for example, those skilled in the art will recognize that the disclosure may be embodied or carried out in a manner that achieves one advantage or a group of advantages as taught herein without necessarily achieving other advantages as may be taught or suggested herein.

Conditional language, such as “can,” “could,” “might,” or “may,” unless specifically stated otherwise, or otherwise understood within the context as used, is generally intended to convey that certain embodiments include, while other embodiments do not include, certain features, elements, and/or steps. Thus, such conditional language is not generally intended to imply that features, elements, and/or steps are in any way required for one or more embodiments or that one or more embodiments necessarily include logic for deciding, with or without user input or prompting, whether these features, elements, and/or steps are included or are to be performed in any particular embodiment.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount.

The scope of the present disclosure is not intended to be limited by the specific disclosures of preferred embodiments in this section or elsewhere in this specification, and may be defined by claims as presented in this section or elsewhere in this specification or as presented in the future. The language of the claims is to be interpreted broadly based on the language employed in the claims and not limited to the examples described in the present specification or during the prosecution of the application, which examples are to be construed as non-exclusive.

1. A surgical tool configured to be used in wrist fusion, or intercarpal fusion, or four corner fusion, the tool comprising:
first and second clamping arms that are each offset relative to a transverse plane defined by a main body of the tool; and
a locking mechanism for locking relative positions of the first and second clamping arms once a desired alignment of bones at a wrist joint is achieved.

2. The surgical tool of claim 1, wherein the first and second clamping arms are offset relative to the transverse plane in opposite directions.

3. The surgical tool of claim 1, wherein the first and second clamping arms are offset relative to the transverse plane such that the tool is configured to be for use by a left-handed user.

4. The surgical tool of claim 1, wherein the first and second clamping arms are offset relative to the transverse plane such that the tool is configured to be for use by a right-handed user.

5. The surgical tool of claim 1, wherein the locking mechanism comprises a screw.

6. A surgical method for performing wrist fusion on a patient using a plate having a curvature configured to substantially follow the natural anatomy of a wrist joint in combination with a reduction tool with offset first and second clamping arms, the method comprising:
attaching the plate to bone(s) at the wrist joint of the patient using at least one screw;
using the reduction tool to reduce the bones at the wrist joint by rotating the offset clamping arms to push a capitate at the wrist joint toward a palm of the patient's hand connected to the wrist joint; and
inserting a remainder of a plurality of screws into the bones at the wrist joint, wherein, when fully implanted, the plurality of screws are coupled to the plate and inserted into the bones at the wrist joint in different directions.

7. The method of claim 6, wherein using the reduction tool is performed before attaching the plate to the bone(s) using at least one screws.

8. The method of claim 6, wherein using the reduction tool is performed after attaching the plate to the bone(s) using at least one screws.

9. The method of claim 6, wherein the first and second clamping arms are offset relative to a transverse plane defined by a main body of the tool in opposite directions.

10. The method of claim 6, wherein the first and second clamping arms of the reduction tool are offset relative to the transverse plane such that the tool is configured to be for use by a left-handed user.

11. The method of claim 6, wherein the first and second clamping arms of the reduction tool are offset relative to the transverse plane such that the tool is configured to be for use by a right-handed user.

12. The method of claim 6, further comprising locking relative positions of the first and second clamping arms once a desired alignment of bones at the wrist joint is achieved using a locking mechanism.

13. The method of claim 12, wherein the locking mechanism comprises a screw.

14. A system for fusing at least two bones in a wrist, or intercarpal fusion, or four corner fusion, the system comprising:
a plate having a curvature configured to substantially follow the natural anatomy of the wrist; and
a plurality of screws configured to point in different directions when coupled to the plate.

15. The system of claim 14, wherein the plurality of screws comprise locking screws.

16. The system of claim 14, wherein the plurality of screws comprise non-locking screws.
